(19) United States
(12) Patent Application Publication
(10) Pub. No.: US 2001/0038804 A1
(43) Pub. Date: Nov. 8, 2001

(54) METHOD OF MAINTAINING THE INTEGRITY OF A WOOD STRUCTURE BY THE USE OF A CHEMICAL TREATMENT AGENT`

(76) Inventor: KENNETH J. NORTON, MADISON, WI (US)

Correspondence Address: FULWIDER PATTON LEE & UTECHT, LLP, 200 OCEANGATE, SUITE 1550, LONG BEACH, CA, 90802, US

(21) Appl. No.: 09/248,397

(22) Filed: Feb. 10, 1999

(51) Int. Cl. A01N 1/00; A61L 9/00

(52) U.S. Cl. 422/28; 422/1; 422/29; 422/31; 422/32; 422/40; 47/57.5; 47/48.5

(57) ABSTRACT

An improved method of maintaining the integrity of a wood structure over a period of years by forming a hole in the wood structure, inserting a container holding the treatment agent and a closure cap into the hole, with such closure cap undergoing an interaction with the vapors of the treatment agent so that the vapors dissolve the closure cap thereby providing an opening through which the treatment agent vapors are released into the internal structure of the wood object, and after the treatment agent vapors are no longer effective to treat the wood, retrieving the container and replacing it with a similar container, treatment agent and closure cap within the same hole to eliminate the necessity of drilling a new hole in the wood structure for such container.

### BACKGROUND OF THE INVENTION

The present invention relates to a method of maintaining the integrity of a wood structure over a period of years by the use of a chemical treatment agent.

Wood structures such as utility poles for electric and telephone transmission wires, pilings for highways, railroad bridges, docks or wharves, and solid or laminated wood building structural components are all subject to attack by wood destroying fungi (wood rot) and insects (termites). Frequently, such attack is unnoticed externally but can be extensive internally. One method of combating such destruction is by the internal application of a chemical agent which is toxic to the wood destroying organisms. Unfortunately, the chemical agent chosen is usually not only toxic to destructive organisms, but is also toxic to man, animals, and the environment as well. The utilization of such chemical treatment agents also presents a severe hazard to personnel who must handle the toxic chemicals. In addition, where wood pilings near waterways are to be the subject of treatment, there is the additional danger of environmental contamination occurring if the treatment agent seeps or spills into the waterway adjacent the piling. Moreover, the conspicuous unattended, application of a chemical fluid to a wood structure over an extended period of time may be interrupted by tampering on the part of children or animals, resulting in their exposure to potentially toxic chemicals.

There are several known devices used to implement the introduction of a chemical treatment agent into the internal environment of a wood structure. These devices enclose the desired toxic chemicals, and when properly inserted into a pre-formed cavity, the chemicals are released by mechanical means. One such example is illustrated by U.S. Pat. No. 3,367,065 wherein the container of toxic material is placed within a prepared cavity located within the interior of a tree. The container is sealed within the tree utilizing a retaining plug having a puncturing means on its inner end. When the retaining plug is impacted into its final position, the end of the container is punctured releasing the toxic contents into the tree interior. There is a danger that the toxic contents can come into contact with personnel installing the device.

U.S. Pat. No. 3,691,683 discloses a method wherein a cartridge which is at least partially filled with the chemical treating agent is knocked into the tree trunk by some mechanical means. This method involves shattering the cartridge end under sufficient impact to generate an overpressure in the cartridge for injecting the chemical treating agent into the tree. Such a shattering technique, however, involves an additional element of risk in the event shattered pieces of the cartridge come in contact with personnel impacting the device.

U.S. Pat. No. 4,344,250 discloses a method wherein the risk factors are significantly reduced. In conducting such method a container of chemical treatment agent is sealed with a material which undergoes an interaction when contacted by the treatment agent or the vapors of such treatment agent. The container is then positioned within a hole bored in the wood structure. After a sufficient period of time has elapsed for the interaction to occur, the closure plug will be dissolved by the treatment agent and/or its vapors to provide an opening through which the treatment agents or its vapors are released into the interior of the wood structure so as to render destructive organisms in the wood harmless.

### SUMMARY OF THE INVENTION

It has been found that while methods such as disclosed in the aforesaid U.S. Pat. No. 4,344,250, are effective for a period of generally eight to ten years, the chemical treatment agent will thereafter lose its toxic effectiveness. Accordingly, it has heretobefore been necessary to drill new holes to receive the chemical treatment agent container and its closure in the wood structure. The forming of such new treatment holes removes additional wood from the wood structure with corresponding loss of structural integrity. In many instances, such loss of structural integrity is not permissible, as for example, where the wood structure is a piling support for highway or railway bridges.

It is a primary object of the present invention to provide an improved method for the internal treatment of a wood structure which permits the re-use of a treatment agent container-receiving hole after the toxicity of the chemical treatment agent of the original container has deteriorated over a period of time.

It is a further object of the present invention to provide a plug for closing the container-receiving hole which remains readily identifiable from the exterior of the wood structure so as to facilitate replacement of the original container.

The method of the present invention also utilizes a retrieval tab that extends from the container through the open-end of the hole so as to facilitate removal of the original container without requiring enlargement of the original container-receiving hole.

These and other features and advantages of the present invention will become apparent from the following detailed description of a preferred embodiment which, taken in conjunction with the accompanying drawings, illustrates by way of example the principles of the invention.

### BRIEF DESCRIPTION OF THE DRAWINGS

FIG. 1 is a perspective view showing a treatment agent container, its closure means and retrieval tab embodying the present invention;

FIG. 2 is a view similar to FIG. 1 but showing a modification of the construction of the container and closure cap;

FIG. 3 is a perspective view of a sealed container disposed within a wood structure;

FIG. 4 is a vertical sectional view of the arrangement of FIG. 3;

FIG. 5 is a perspective view similar to FIG. 4 after the closure cap has been dissolved by the chemical treatment agent;

FIG. 6 is a view similar to FIGS. 4 and 5 showing the container and closure cap of FIG. 4 disposed within a hole in a wooden structure wherein the hole is provided with a restraining cork to prevent the container from falling out of the hole as the plug is secured within the opening to the hole;

FIG. 7 is a sectional view taken in enlarged scale along line 7-7 of FIG. 4;

FIG. 8 is a broken side elevational view showing a plug being removed from the opening of the hole; and

FIG. 9 is a view similar to FIG. 8 showing the plug removed from the opening of the hole.

### DETAILED DESCRIPTION OF THE PREFERRED EMBODIMENTS

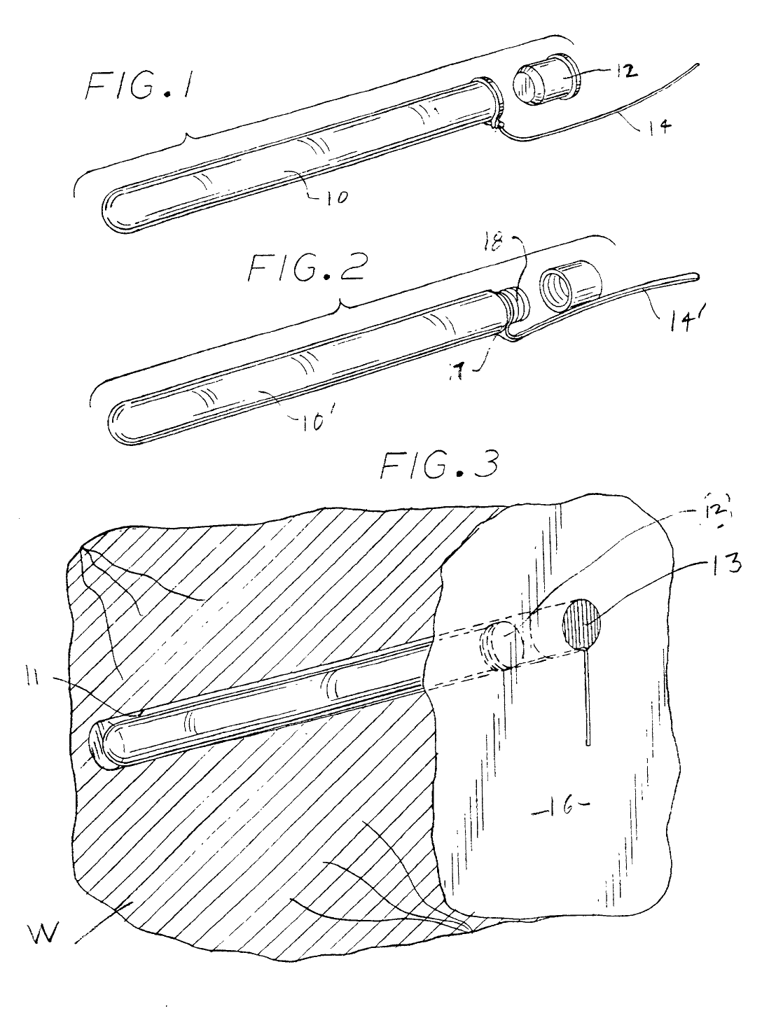

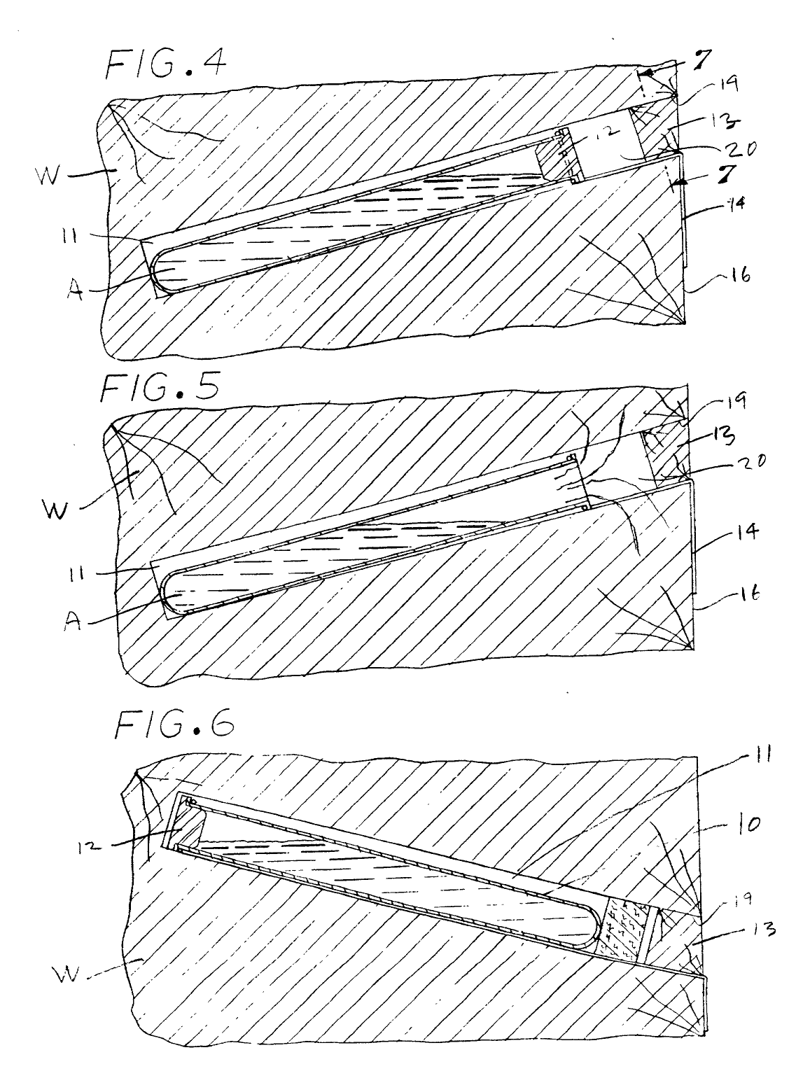

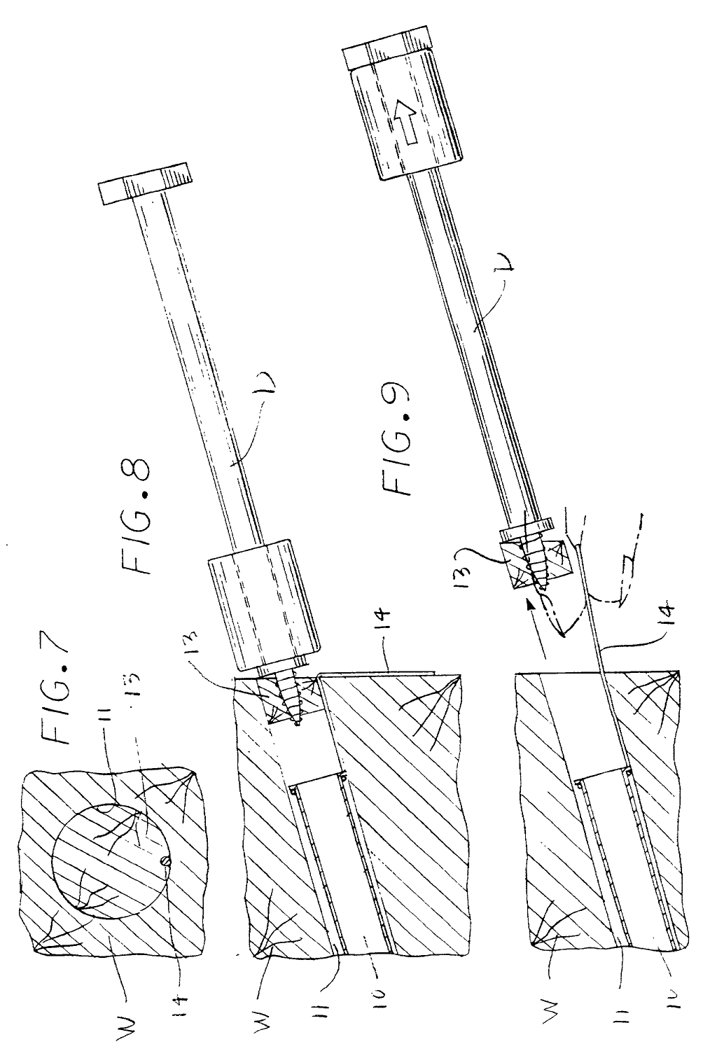

Referring to the drawings and particularly FIGS. 1, 3, 4 and 5, the method of treatment of a wood structure W according to the present invention utilizes a container 10 of any desired size and shape disposed within a hole 11 formed in the wood structure. The dimensions are governed by the requirement by the specific application to the wood structure. The container 10 is open at its upper end to removably receive a closure cap 12 which has a press fit with the open end of the container so as to form a stopper for the open-end of the container which fits flush against the inner circumference of the container end section to provide an essentially airtight seal at the open end of the container. A body of a chemical treatment agent A is confined with the container 10 by cap 12. The container 10 is sealed within hole 11 by a wood plug 13. A retrieving tab 14 secured to the upper end of the container extends alongside the cap and upwardly out of the hole so as to be accessible from the outer surface 16 of the wood structure W. Retrieving tab 14 should be formed of a material which will maintain its strength over a period of years, as for example, stainless steel.

Critical to the successful utilization of this treatment method is the selection of the proper combination of materials used to fabricate both the container and the closure cap in relation to the specific chemical properties of each individual component or combination of components used in the treatment agent to be enclosed therein. In a preferred embodiment, hollow container 10 is formed of a material which is resistant to chemical degradation or solubilization by the treatment agent A or its vapors, while the closure cap 12 is formed out of a material which undergoes an interaction with the vapors of the treatment agent. The type of interaction which occurs will depend on the specific chemical ingredients which are utilized in each combination. At times the interaction will be a dissolution reaction wherein the cap 12 is soluble in the treatment solution. In other cases the interaction will involve a chemical reaction wherein the cap 12 undergoes a degradation or disintegration process. When the proper combination of materials is used, the container 10 is filled or partially filled with the desired chemical treatment agent A, sealed, and thereafter placed at the treatment site within hole 11, either the treatment agent itself or its vapors spontaneously begin to dissolve or disintegrate the cap 12, thereby creating an opening or passage out of the container through which the vapors of the chemical treatment are released into the interstices of the wood.

The selection of materials to be used in fabricating the container and its cap will vary depending upon the exact chemical properties of the particular treatment agent. If several chemicals are to be combined or if a solvent or diluent is to be added, then the chemical properties of the additional ingredients must also be considered. The chemical treatment composition may be a water soluble mixture, or it may be an emulsion comprised of agents such as fungicides, insecticides or plant nutrient compositions commonly used for treating trees and other wood structures. The selection of a suitable chemically soluble or degradeable material for use in making the cap could be determined by a process of trial and error, and in all such instances materials may not as yet be known or available.

Container 10 may be formed of any suitable thin or thick walled material but in the preferred form, it comprises a clear, rigid material which permits the determination of the amount of chemical treatment agent contained therein to be determined by visual inspection. Such material should be essentially non-reactive and non-soluble when in contact with any component of the chemical treatment agent or its vapors. Examples of suitable materials are the following: glass, or solvent/chemical resistant plastics such as, but not limited to, acetal homopolymers, diallylphthalate, cellulose acetate, nylon, polyethylene, polypropylene and polyethylene/ethylene copolymers.

The chemically reactive closure cap must be fabricated out of a suitable material capable of undergoing dissolution or degradation in the liquid or vapor phase of one or more the components of the chemical treatment composition. Plastics, such as, but not limited to the following, are examples of suitable fabrication materials for the cap: acrylics, cellulosic materials such as cellulose acetate butyrate and cellulose propionate, polycarbonate, and polystryene for organic treatment solutions, and polyvinyl alcohol for water based treatment solutions.

Many chemical treating agents and fumigants are known which are suitable for use with the container 10 and its cap 12. Examples of such agents include: Sodium N-methyldithiocarbamate, methyl isothiocyanate and methyl isothiocynate combined with chlorinated C₃ hydrocarbons, cis-N-[1,1,2,2, tetrachloroethyl)thio]-4-cyclohexene-1,2-dicarboximide, chloropicrin (trichloronitromethlane), aluminum phosphide, carbon disulfide, carbon tetrachloride, 1,3-dichloropropene and mixtures of 1,3-dichloropropene and 1,2-dichloropropene, dichlorethyl ether, ethylene dibromide, ethylene dichloride hydrocyanic acid, orthodichlorobenzene, acrylonitrile, 3-chloro-2-methylpropene, trichloroethylene, 1,1,1, trichloroethane, monochloroacetonitrile, trichloracetonitrile, 1,2-dicloropropane, 1,2 dibromo-3-chloropropane, 1-bromo-2-chloroethane, 3-bromo-1-chloropropene, 3-bromo-1-propyne, 1-chloro-2,3-epoxypropane, 1,2-dichloroethane, and tetrachloroethylene.

A large number of different solvents may be used for the preparation of solutions containing the above chemical treatment agents. The only requirement in choosing a solvent is that it be inert toward the chemical treatment agents. The treatment agent may often be utilized in pure, undiluted form, or the treatment agent concentration may have as its upper limit the solubility of the chemical treating agent (or other ingredient of the chemical combination) in the particular solvent or solvent mixture used. However, the active treatment chemicals may be present over a wide range of concentrations below this limit without effecting the operability of the treatment method utilizing the chemically reactive closure described herein. Although the concentration of the active ingredient is not critical to the present method of the present invention, the rate of the chemical interaction may be enhanced when high concentrations of the chemical agents are used.

In addition, the rate of the reaction between the closure means and the chemical agent, is partially dependent upon the physical characteristics of the closure means employed. For example, decreasing the radial dimensions or increasing the thickness of the closure will slow down the release of the vapors of the treatment agent A from the container 10. By varying these dimensions, it is possible effectively control the release of the chemical treatment agent to ensure the safety of all those persons who must come into contact with the container prior to treatment.

U.S. Pat. Nos. 3,113,908 and 3,265,561 assigned to Schering A. G., Berlin, Germany, and U.S. Pat. Nos. 3,205,129 and 3,240,666 assigned to Morton Salt Company, Chicago, Ill. disclose various examples of typical solutions, mixtures or emulsions of known chemical fumigant pesticide agents combined with a variety of solvents, many of which would be suitable as a treatment agent for use in carrying out the method of the instant invention.

Referring now to FIG. 2, there is illustrated an alternate embodiment of the container 10′ and its closure cap 12′. In this embodiment, a neck 18 formed on the open end of the container 10′ is externally threaded so as to receive the complimentary internal threads of hollow cap 12′. In this embodiment, the retrieving tab 14′ Includes an apertured flap which encompasses the threaded neck 18 of the container and is secured to such container by screwing the cap 12′ onto the external threads of the container.

A preferred chemical treatment agent A utilized with the method of the present invention may be of the type described in the aforementioned U.S. Pat. No. 4,344,250 i.e. mixtures or emulsions of known chemical fumigant pesticide agents combined with a variety of solvents. It has been found that TIMBERFUME (chloropicrin) sold by OSMOSE, INC. of Buffalo, N.Y., is suitable as a chemical treatment agent for use in carrying out the method of the present invention.

Referring to FIGS. 3, 4, and 5, in carrying out the method of the present invention, hole 11 may be formed by any suitable drill-type device and should support the container wholly within the lateral confines of the wood structure W. When the container 10 has been deposited within the hole 11 through hole opening 19 its tab 14 will extend through the hole and thereafter lie flush against the exterior surface 16 of the wood structure W. A space 20 is defined in the upper portion of the hole 11 between the upper end of the container 10 and plug 12. The plug may be made of any suitable material, such as wood, cork or resilient synthetic plastic. When the plug is forced into the open end of hole 11 it will effectively seal the interior of the hole from the ambient atmosphere. The plug confines the vapors of chemical treatment A to the interstices of the wood structure W.

FIG. 5 discloses the appearance of the container 10 after the chemical treatment agent vapors have dissolved cap 12. It will be understood that even before the cap C is entirely dissolved its partial dissolution will permit vapors from the chemical treatment agent to pass out of the open end of the container and space 20 and then into the interstices of the wood structure. The rate of reaction between the material of the cap 12 and the chemical treatment agent A is partially dependent of the physical characteristics of the cap.

In the embodiment of the invention shown in FIGS. 1-5, the hole 11 slants downwardly from the exterior 16 of the wooden structure W and the bottom of the container is inserted into hole 11 with the cap 12 positioned higher than the closed end of the container. This arrangement insures that none of the chemical treatment agent A can spill out of the hole 11 so as to leak into any surrounding water or soil since such leakage would cause risk of toxic injury to fish and wildlife.

Referring now to FIG. 6, the hole 11 is shown slanting upwardly from the exterior surface 16 of the wood structure W. With this hole inclination, it is necessary to restrain the container C against sliding out of the hole as the plug 13 is secured within the opening of the hole. This can be accomplished by inserting a cork 22 through the opening 19 of hole 11 so as to engage the lower end of the container C and hold it in place as plug 13 is pounded into the opening of the hole. The cork 22 will also absorb shocks to protect the container against breakage as the plug 13 is pounded into the opening of the hole. The cork 22 also absorbs any vibrations which might result from train traffic if the wood structure W forms part of a railroad bridge.

It should be particularly noted that the surface of the plug 13 exposed to the exterior of the wood structure W should preferably be brightly colored. This feature greatly facilitates locating the hole 11 when it becomes necessary to replace the container 10. Such replacement becomes necessary when the chemical treatment agent A has deteriorated to the extent that its toxic vapors are no longer effective in treating the wood structure.

Referring now to FIGS. 8 and 9 when the container C is to be removed from hole 11, a conventional auger type drill D is inserted into the plug 13 and actuated so as to withdraw the plug from the opening 19 of hole 11. Thereafter, tab 14 is manually engaged and utilized to pull the empty container out of the hole. If the container C is structurally sound, it will be refilled with a chemical treatment agent A and reinserted into the hole in the same manner it was initially installed. Alternatively, a new container will be utilized. By reusing the hole 11 the necessity of drilling new container-receiving holes with a consequent weakening of the wood structure W is avoided. In this manner the service life of the wood structure can be significantly increased. Moreover, the costs of drilling new holes is eliminated.

While a particular form of the invention has been illustrated and described, it will also be apparent to those skilled in the art that various modifications can be made without departing from the spirit and scope of the invention. Accordingly, it is not intended that the invention be limited except by the appended claims.

What is claimed is:

1. A method of maintaining the integrity of a wood structure over a period of years by the use of a chemical treatment agent, which includes the steps of:
forming a hole in the wood structure;
placing the treatment agent in a container having an open end, which container is fabricated out of a material that is substantially non-reactive when placed in contact with the treating agent or the vapors of the treating agent;
sealing the open end of the container with a closure cap substantially fabricated out of a material which undergoes an interaction when contacted with the treatment agent or the vapors of the treatment agent such that after a sufficient period of time has elapsed for the interaction to occur, an opening will spontaneously be provided through the cap to permit the vapors of the treating agent to pass out of the container and into the wood structure;
closing the entrance to the hole with a plug that remains readily identifiable from the exterior surface of the wood structure;
removing the plug from the entrance of the hole after the toxicity of the vapors of the treatment agent has substantially dissipated;
retrieving the container from the hole;
replacing the original container with a similar container, treatment agent and closure cap; and
again closing the hole with a plug that remains readily identifiable from the exterior surface of the wood structure.

2. A method as set forth in claim 1, wherein the plug is brightly colored.

3. A method as set forth in claim 1, wherein the container is provided with a retrieval tab that extends from the open end of the hole.

4. A method as set forth in claim 2, wherein the container is provided with a retrieval tag that extends from the open end of the hole.
